(19) United States
(12) Patent Application Publication
(10) Pub. No.: US 2025/0225763 A1
(43) Pub. Date: Jul. 10, 2025

(54) MEDICAL IMAGE ANALYSIS APPARATUS, MEDICAL IMAGE ANALYSIS METHOD, AND PROGRAM

(71) Applicant: FUJIFILM Corporation, Tokyo (JP)

(72) Inventors: Taro HATSUTANI, Tokyo (JP); Akimichi ICHINOSE, Tokyo (JP)

(73) Assignee: FUJIFILM Corporation, Tokyo (JP)

(21) Appl. No.: 19/090,355

(22) Filed: Mar. 25, 2025

Related U.S. Application Data
(63) Continuation of application No. PCT/JP2023/032971, filed on Sep. 11, 2023.

(30) Foreign Application Priority Data
Sep. 28, 2022 (JP) 2022-154572

(51) Int. Cl. G06V 10/25; A61B 6/03; G06V 10/774; G06V 30/10; G16H 30/20

(52) U.S. Cl. CPC G06V 10/25; G06V 10/774; G06V 30/10; G16H 30/20; A61B 6/032; G06V 2201/03

(57) ABSTRACT

A medical image analysis apparatus, a medical image analysis method, and a program for specifying a region of interest intended by a doctor in a medical image of a source of creation of a key image are provided. A medical image analysis apparatus includes at least one processor, and at least one memory in which an instruction to be executed by the at least one processor is stored, in which the at least one processor is configured to acquire a key image that is created from a medical image and that includes a region of interest, extract association information between the key image and the medical image of a source of creation of the key image by analyzing the key image, and specify the region of interest in the medical image based on the association information.

### CROSS-REFERENCE TO RELATED APPLICATIONS

The present application is a Continuation of PCT International Application No. PCT/JP2023/032971 filed on Sep. 11, 2023 claiming priority under 35 U.S.C § 119(a) to Japanese Patent Application No. 2022-154572 filed on Sep. 28, 2022. Each of the above applications is hereby expressly incorporated by reference, in its entirety, into the present application.

### BACKGROUND OF THE INVENTION

### 1. Field of the Invention

The present invention relates to a medical image analysis apparatus, a medical image analysis method, and a program, and particularly to a technique for using a key image for training a learning model.

### 2. Description of the Related Art

Hospitals have a large number of key images created in a case where a doctor interprets a medical image. The key image is a representative image indicating a region of interest. For example, the region of interest is a lesion. In a case where the original image is a three-dimensional medical image such as a CT image and an MRI image, the key image may be stored as a slice of the region of interest, may be stored by further cropping the slice, or may be stored by attaching an annotation such as a rectangle or an arrow to the region of interest.

In the key image, a positional relationship with respect to the original image may be missing when the key image is created, or image information may be defected by addition of the annotation or the like when the key image is created.

JP2020-28583A discloses a technique for acquiring a key image, acquiring a cross-sectional image parallel to the key image, and generating a supplementary image. JP2015-156898A discloses a technique for analyzing a key image, separating the key image into a medical image and an annotation image, and acquiring medical image information corresponding to the medical image.

### SUMMARY OF THE INVENTION

Training a deep learning model requires a large amount of data. Thus, use of the key image is considered. However, a problem arises in that which position of the original image corresponds to the key image is not known. For example, which position of which slice the key image is created from is not clear. In a case where, for example, the key image does not have an annotation, or the key image has only an arrow annotation, a problem arises in that a computer cannot determine the region of interest intended by the doctor.

The present invention has been conceived in view of such circumstances, and an object of the present invention is to provide a medical image analysis apparatus, a medical image analysis method, and a program for specifying a region of interest intended by a doctor in a medical image of a source of creation of a key image.

In order to achieve the object, according to a first aspect of the present disclosure, there is provided a medical image analysis apparatus comprising at least one processor, and at least one memory in which an instruction to be executed by the at least one processor is stored, in which the at least one processor is configured to acquire a key image that is created from a medical image and that includes a region of interest, extract association information between the key image and the medical image of a source of creation of the key image by analyzing the key image, and specify the region of interest in the medical image based on the association information. According to the present aspect, the region of interest intended by a doctor can be specified in the medical image of the source of creation of the key image. Thus, the medical image in which the region of interest is specified can be used as training data of a learning model that estimates the region of interest from the medical image.

According to a second aspect of the present disclosure, in the medical image analysis apparatus according to the first aspect, the at least one processor is preferably configured to estimate the region of interest from the key image, and add the estimated region of interest to the medical image.

According to a third aspect of the present disclosure, in the medical image analysis apparatus according to the first or second aspect, it is preferable that the key image includes an annotation indicating the region of interest, and the at least one processor is configured to add the annotation to the medical image, and specify the region of interest in the medical image based on the added annotation.

According to a fourth aspect of the present disclosure, in the medical image analysis apparatus according to the third aspect, the at least one processor is preferably configured to detect the annotation from the key image.

According to a fifth aspect of the present disclosure, in the medical image analysis apparatus according to any one of the first to fourth aspects, the medical image preferably includes at least one of a two-dimensional static image, a three-dimensional static image, or a video.

According to a sixth aspect of the present disclosure, in the medical image analysis apparatus according to any one of the first to fifth aspects, the key image is preferably a result of volume rendering created from the medical image.

According to a seventh aspect of the present disclosure, in the medical image analysis apparatus according to any one of the first to sixth aspects, it is preferable that the at least one processor is configured to extract the association information by analyzing a character in the key image using character recognition, and the association information includes at least one of a window width, a window level, a slice number, or a series number of the key image.

According to an eighth aspect of the present disclosure, in the medical image analysis apparatus according to any one of the first to seventh aspects, it is preferable that the at least one processor is configured to extract the association information by performing image recognition of the key image, and the association information includes at least one of a window width, a window level, or an annotation of the key image.

According to a ninth aspect of the present disclosure, in the medical image analysis apparatus according to any one of the first to eighth aspects, the at least one processor is preferably configured to extract the association information from a result of registration between the medical image and the key image.

According to a tenth aspect of the present disclosure, in the medical image analysis apparatus according to any one of the first to ninth aspects, the at least one processor is preferably configured to estimate a position corresponding to the key image in the medical image based on the association information.

According to an eleventh aspect of the present disclosure, in the medical image analysis apparatus according to any one of the first to tenth aspects, the region of interest is preferably at least one of a mask, a bounding box, or a heat map.

According to a twelfth aspect of the present disclosure, in the medical image analysis apparatus according to any one of the first to eleventh aspects, the medical image is preferably a digital imaging and communications in medicine (DICOM) image.

In order to achieve the object, according to a thirteenth aspect of the present disclosure, there is provided a medical image analysis method comprising acquiring a key image that is created from a medical image and that includes a region of interest, extracting association information between the key image and the medical image of a source of creation of the key image by analyzing the key image, and specifying the region of interest in the medical image based on the association information. According to the present aspect, the region of interest intended by a doctor can be specified in the medical image of the source of creation of the key image. Thus, the medical image can be used as training data of a learning model.

In order to achieve the object, according to a fourteenth aspect of the present disclosure, there is provided a program causing a computer to execute the medical image analysis method according to the thirteenth aspect. The present disclosure also includes a non-transitory computer-readable recording medium, such as a compact disk-read only memory (CD-ROM), storing the program according to the fourteenth aspect.

According to the present invention, the region of interest intended by the doctor in the medical image of the source of creation of the key image can be specified.

### BRIEF DESCRIPTION OF THE DRAWINGS

FIG. 1 is an overall configuration diagram of a medical image analysis system.

FIG. 2 is a block diagram illustrating an electric configuration of a medical image analysis apparatus.

FIG. 3 is a block diagram illustrating a functional configuration of the medical image analysis apparatus.

FIG. 4 is a flowchart illustrating a medical image analysis method according to a first embodiment.

FIG. 5 is a diagram illustrating a key image and a medical image of a source of creation of the key image.

FIG. 6 is a flowchart illustrating a medical image analysis method according to a second embodiment.

FIG. 7 is a diagram illustrating an example of the key image.

FIG. 8 is a flowchart illustrating a medical image analysis method according to a third embodiment.

### DESCRIPTION OF THE PREFERRED EMBODIMENTS

Hereinafter, preferable embodiments of the present invention will be described in detail with reference to the accompanying drawings.

### Medical Image Analysis System

A medical image analysis system according to the present embodiment is a system that specifies a region of interest in a medical image of a source of creation from a key image created by a doctor. The medical image of the source of creation in which the region of interest is specified can be used as training data of a learning model.

FIG. 1 is an overall configuration diagram of a medical image analysis system 10. As illustrated in FIG. 1, the medical image analysis system 10 is composed of a medical image examination apparatus 12, a medical image database 14, a user terminal apparatus 16, an interpretation report database 18, and a medical image analysis apparatus 20.

The medical image examination apparatus 12, the medical image database 14, the user terminal apparatus 16, the interpretation report database 18, and the medical image analysis apparatus 20 are connected to each other to be capable of transmitting and receiving data through a network 22. The network 22 includes a wired or wireless local area network (LAN) for communication and connection of various apparatuses in a medical institution. The network 22 may include a wide area network (WAN) for connecting LANs of a plurality of medical institutions to each other.

The medical image examination apparatus 12 is an imaging apparatus that generates a medical image by imaging an examination target part of an object to be examined. Examples of the medical image examination apparatus 12 include an X-ray imaging apparatus, a computed tomography (CT) apparatus, a magnetic resonance imaging (MRI) apparatus, a positron emission tomography (PET) apparatus, an ultrasound apparatus, a computed radiography (CR) apparatus using a planar X-ray detector, and an endoscope apparatus.

The medical image database 14 is a database for managing the medical image captured by the medical image examination apparatus 12. A computer comprising a high-capacity storage device for storing the medical image is applied as the medical image database 14. The computer incorporates software that provides a function of a database management system.

The medical image may be a two-dimensional static image or a three-dimensional static image captured by an X-ray imaging apparatus, a CT apparatus, an MRI apparatus, or the like or may be a video captured by an endoscope apparatus.

A digital imaging and communications in medicine (DICOM) standard can be applied as a format of the medical image. Accessory information (DICOM tag information) defined in the DICOM standard may be added to the medical image. The term “image” in the present specification includes not only a meaning of the image itself such as a photo but also a meaning of image data that is a signal indicating the image.

The user terminal apparatus 16 is a terminal apparatus with which a doctor creates and views an interpretation report. For example, a personal computer is applied as the user terminal apparatus 16. The user terminal apparatus 16 may be a workstation or may be a tablet terminal. The user terminal apparatus 16 comprises an input device 16A and a display 16B. The doctor inputs an instruction to display the medical image using the input device 16A. The user terminal apparatus 16 displays the medical image on the display 16B. The doctor creates the interpretation report by interpreting the medical image displayed on the display 16B, creating the key image from the medical image using the input device 16A, and inputting a medical opinion that is an interpretation result.

The key image is an image in which information about the doctor is input. The key image is an image associated with the medical image of the source of creation at a patient level and an imaging date and time level. However, the key image is an image in which information about a positional relationship with respect to the medical image of the source of creation is missing. The key image may be an image in which an information amount is reduced from that of the medical image of the source of creation by converting the medical image of the source of creation into an image of bitmap or the like, or may be an image after conversion into an image in which the information amount is not reduced. The key image may be an image in which image information at a position to which an annotation is added is missing in image information of the original medical image. The key image may be a result of volume rendering created from the medical image.

The key image includes a region of interest in which the doctor is interested. The key image may include an annotation indicating the region of interest. The annotation of the key image may be at least one of a circle, a rectangle, an arrow, a line segment, a point, or a scribble.

The key image may include character information. The character information may include at least one of a window width, a window level, a slice number, or a series number of the key image.

The interpretation report database 18 is a database for managing the interpretation report generated by a user in the user terminal apparatus 16. The interpretation report includes the key image. A computer comprising a high-capacity storage device for storing the interpretation report is applied as the interpretation report database 18. The computer incorporates software that provides the function of the database management system. The medical image database 14 and the interpretation report database 18 may be composed of one computer.

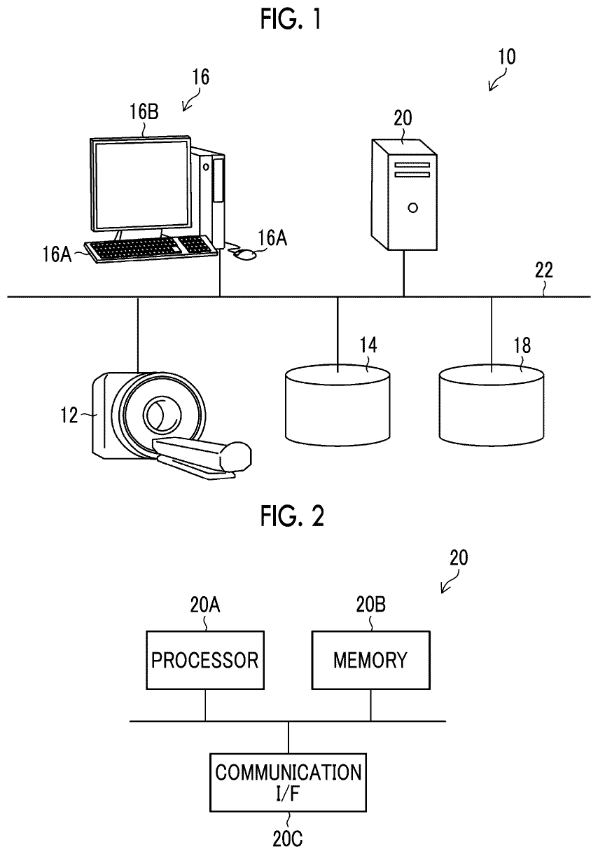

The medical image analysis apparatus 20 is an apparatus that specifies the region of interest in the medical image. A personal computer or a workstation (an example of a “computer”) can be applied as the medical image analysis apparatus 20. FIG. 2 is a block diagram illustrating an electric configuration of the medical image analysis apparatus 20. As illustrated in FIG. 2, the medical image analysis apparatus 20 comprises a processor 20A, a memory 20B, and a communication interface 20C.

The processor 20A executes an instruction stored in the memory 20B. A hardware structure of the processor 20A includes the following various processors. The various processors include a central processing unit (CPU) that is a general-purpose processor operating as various functional units by executing software (a program), a graphics processing unit (GPU) that is a processor specialized in image processing, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of two or more processors of the same type or different types (for example, a plurality of FPGAs, a combination of a CPU and an FPGA, or a combination of a CPU and a GPU). A plurality of functional units may be composed of one processor. A first example of the plurality of functional units composed of one processor is, as represented by a computer such as a client or a server, a form of one processor composed of a combination of one or more CPUs and software, in which the processor operates as the plurality of functional units. A second example is, as represented by a system on chip (SoC) or the like, a form of using a processor that implements functions of the whole system including the plurality of functional units in one integrated circuit (IC) chip. Various functional units are configured using one or more of the various processors as a hardware structure.

The hardware structure of the various processors is more specifically an electric circuit (circuitry) in which circuit elements such as semiconductor elements are combined.

The memory 20B stores the instruction to be executed by the processor 20A. The memory 20B includes a random access memory (RAM) and a read only memory (ROM) (not illustrated). The processor 20A executes various types of processing of the medical image analysis apparatus 20 by executing software in the RAM as a work region using various programs and parameters including a medical image analysis program (described later) stored in the ROM, and using the parameters stored in the ROM or the like.

The communication interface 20C controls communication with the medical image examination apparatus 12, the medical image database 14, the user terminal apparatus 16, and the interpretation report database 18 through the network 22 in accordance with a predetermined protocol.

The medical image analysis apparatus 20 may be a cloud server accessible from the plurality of medical institutions through the Internet. Processing performed in the medical image analysis apparatus 20 may be a paid or fixed-rate cloud service.

### Functional Configuration as Medical Image Analysis Apparatus

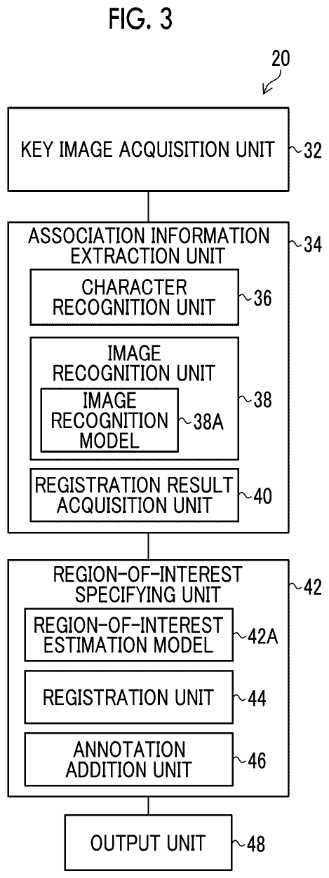

FIG. 3 is a block diagram illustrating a functional configuration of the medical image analysis apparatus 20. Each function of the medical image analysis apparatus 20 is realized by executing the program stored in the memory 20B via the processor 20A. As illustrated in FIG. 3, the medical image analysis apparatus 20 comprises a key image acquisition unit 32, an association information extraction unit 34, a region-of-interest specifying unit 42, and an output unit 48.

The key image acquisition unit 32 acquires the key image including the region of interest from the interpretation report database 18.

The association information extraction unit 34 extracts association information between the key image and the medical image of the source of creation of the key image by analyzing the key image. That is, the association information is information for associating the key image with the medical image of the source of creation of the key image. For example, the association information is information captured in the key image separately from a subject. For example, the association information includes at least one of a series number, a slice number, a window width, a window level, or an annotation of the medical image of the source of creation of the key image. The association information may be a result of registration between the key image and the medical image of the source of creation of the key image. The association information extraction unit 34 includes a character recognition unit 36, an image recognition unit 38, and a registration result acquisition unit 40.

The character recognition unit 36 extracts the association information by analyzing a character in the key image using a well-known character recognition technique such as optical character recognition (OCR). The association information extracted by the character recognition unit 36 may include at least one of the window width, the window level, the slice number, or the series number of the key image.

The image recognition unit 38 extracts the association information by performing image recognition of the key image. The association information extracted by the image recognition unit 38 may include at least one of the window width, the window level, or the annotation of the key image. The image recognition unit 38 comprises an image recognition model 38A. The image recognition model 38A that extracts the window width or the window level of the key image is a classification model or a regression model using a convolution neural network (CNN). The image recognition model 38A that recognizes the annotation of the key image is a segmentation model or a detection model to which a convolutional neural network is applied. The image recognition unit 38 may comprise a plurality of image recognition models 38A among the classification model, the regression model, the segmentation model, and the detection model. The image recognition model 38A is stored in the memory 20B.

The image recognition unit 38 detects the annotation added to the key image. The annotation to be detected by the image recognition unit 38 may include at least one of a circle, a rectangle, an arrow, a line segment, a point, or a scribble.

The registration result acquisition unit 40 acquires the result of registration between the key image and the medical image via a registration unit 44 (described later).

The region-of-interest specifying unit 42 specifies the region of interest based on the association information extracted by the association information extraction unit 34. Using the association information, for example, the region-of-interest specifying unit 42 first estimates a position corresponding to the key image in the medical image of the source of creation of the key image and then specifies the region of interest in the medical image.

The region-of-interest specifying unit 42 may specify the region of interest from a two-dimensional image or may specify the region of interest from a three-dimensional image. The region of interest to be specified may be a two-dimensional region or may be a three-dimensional region.

The region-of-interest specifying unit 42 includes a region-of-interest estimation model 42A, the registration unit 44, and an annotation addition unit 46. The region-of-interest estimation model 42A is a deep learning model that outputs a position of the region of interest in an input image in a case where an image is provided as input. The region-of-interest estimation model 42A may be a trained model to which a CNN is applied. The region-of-interest estimation model 42A is stored in the memory 20B.

The registration unit 44 performs registration between the key image and the medical image of the source of creation of the key image. Registration between the key image and the medical image of the source of creation of the key image means association between respective pixels of both images showing the same subject such as an organ. The result of registration between the key image and the medical image via the registration unit 44 includes a correspondence relationship between the pixels of the key image and the pixels of the medical image. The annotation addition unit 46 adds an annotation to the medical image of the source of creation of the key image.

The output unit 48 outputs the region of interest specified by the region-of-interest specifying unit 42 to record the region of interest in a learning database (not illustrated). The region of interest to be output may be at least one of a mask, a bounding box, or a heat map assigned to the medical image of the source of creation of the key image.

### Medical Image Analysis Method: First Embodiment

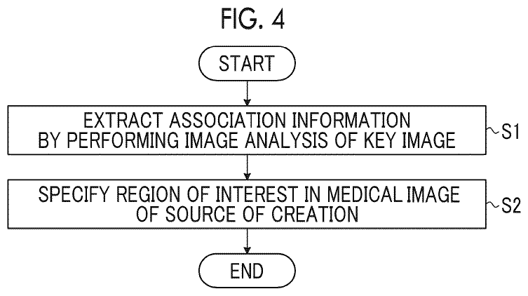

FIG. 4 is a flowchart illustrating a medical image analysis method according to a first embodiment using the medical image analysis apparatus 20. The medical image analysis method is a method of specifying the region of interest in the medical image of the source of creation of the key image. The medical image analysis method is implemented by executing the medical image analysis program stored in the memory 20B via the processor 20A. The medical image analysis program may be provided by a non-transitory computer-readable storage medium or may be provided through the Internet.

In step S1, the key image acquisition unit 32 acquires the key image from the interpretation report database 18. The key image acquisition unit 32 may acquire the key image from a source other than the interpretation report database 18 through the network 22. The association information extraction unit 34 extracts the association information required for associating the key image with the medical image of the source of creation of the key image by performing image analysis of the acquired key image. The image analysis includes character recognition and image recognition.

Next, in step S2, the region-of-interest specifying unit 42 specifies the region of interest in the medical image of the source of creation of the key image based on the association information extracted in step S1.

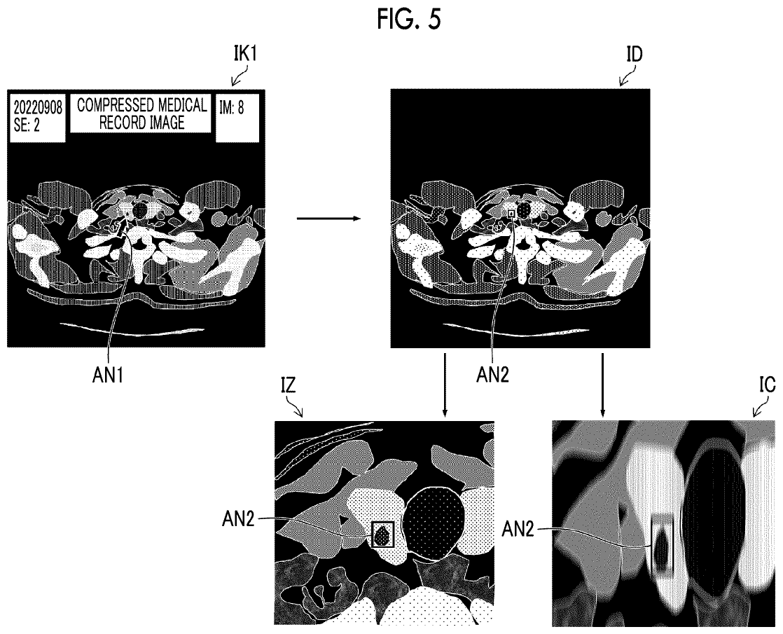

FIG. 5 is a diagram illustrating the key image and the medical image of the source of creation of the key image. A key image IK1 illustrated in FIG. 5 is a two-dimensional image. The key image IK1 includes character information of “20220908”, “SE:2”, “COMPRESSED MEDICAL RECORD IMAGE”, and “IM:8”. The character recognition unit 36 extracts at least one of the window width, the window level, the slice number, or the series number of the key image IK1 as the association information by recognizing the characters.

The key image IK1 includes an arrow annotation AN1. The image recognition unit 38 extracts the annotation AN1 as the association information by performing image recognition of the key image IK1. The image recognition unit 38 may extract at least one of the slice number, the series number, the window width, or the window level as the association information by performing image recognition of the key image IK1.

A medical image ID illustrated in FIG. 5 is a three-dimensional image of the source of creation of the key image IK1 and is an image in which a rectangle annotation AN2 is added to the region of interest specified by the region-of-interest specifying unit 42.

An enlarged image IZ illustrated in FIG. 5 is an image obtained by enlarging the region to which the annotation AN2 is added in the medical image ID. A coronal image IC illustrated in FIG. 5 is an image of a coronal cross section including the region to which the annotation AN2 is added in the medical image ID. The region of interest in the medical image can be three-dimensionally specified by specifying the region of interest in the three-dimensional medical image of the source of creation of the key image. Accordingly, various types of images including the region of interest can be created. Thus, the medical image in which the region of interest is specified can be used as the training data of the learning model that extracts the region of interest from the image.

### Medical Image Analysis Method: Second Embodiment

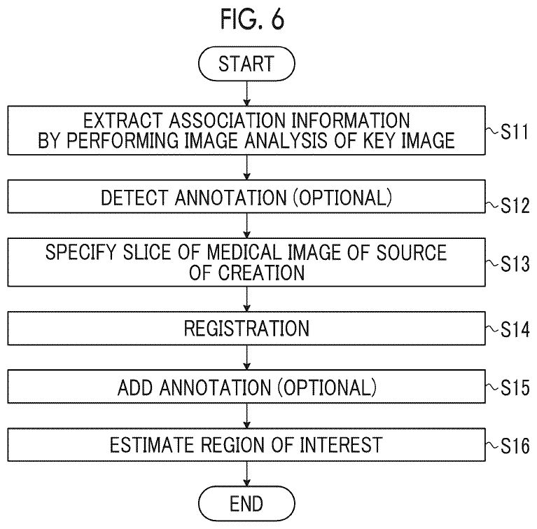

FIG. 6 is a flowchart illustrating a medical image analysis method according to a second embodiment.

Step S11 is the same as step SI of the first embodiment. The image recognition unit 38 extracts the association information from the key image using the image recognition model 38A. The character recognition unit 36 extracts the association information from the key image using OCR.

In step S12, in a case where an annotation is added to the key image acquired in step S11, the image recognition unit 38 detects the annotation from the key image.

In step S13, the region-of-interest specifying unit 42 specifies a slice image of the medical image of the source of creation at the same position as the key image based on the slice number in the association information extracted in step S11. In a case where the slice number cannot be extracted in step S11, the slice image at the same position as the key image is specified using a well-known method.

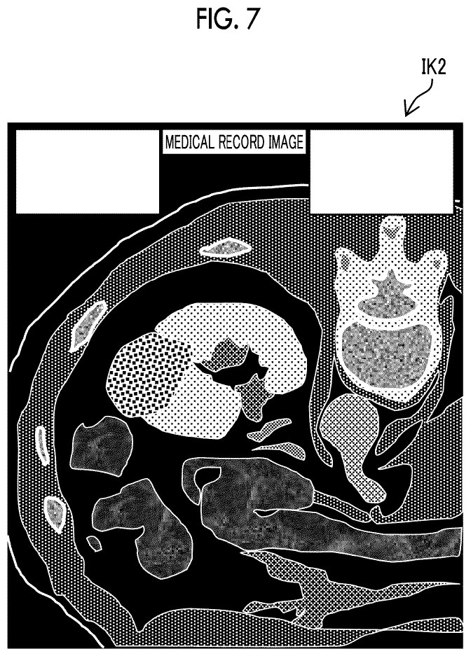

In step S14, the registration unit 44 performs registration between the key image and the slice image specified in step S13. The key image may be cropped or rotated from the slice image of the medical image of the source of creation. Thus, the key image may require registration. FIG. 7 is a diagram illustrating an example of the key image. A key image IK2 illustrated in FIG. 7 is a cropped key image not having an annotation.

In step S15, in a case where an annotation is added to the key image acquired in step S11, the annotation addition unit 46 adds the annotation to the slice image specified in step S13. The registration in step S14 enables the annotation addition unit 46 to add the annotation to the same position of the slice image as a position of the annotation of the key image.

In step S16, the region-of-interest specifying unit 42 specifies the region of interest in the slice image based on the annotation added in step S15. The region-of-interest specifying unit 42 specifies the region of interest using the region-of-interest estimation model 42A. A result of specifying the region of interest may be at least one of a mask, a bounding box, or a heat map. The output unit 48 outputs the specified region of interest.

By adding the annotation of the key image to the slice image of the source of creation of the key image and estimating the region of interest based on the annotation, the region of interest in the slice image can be specified. Accordingly, the region of interest in the medical image of the source of creation can be specified.

While addition of the annotation to the key image acquired in step S11 is described, the region-of-interest estimation model 42A can also estimate the region of interest from the key image not including the annotation.

### Medical Image Analysis Method: Third Embodiment

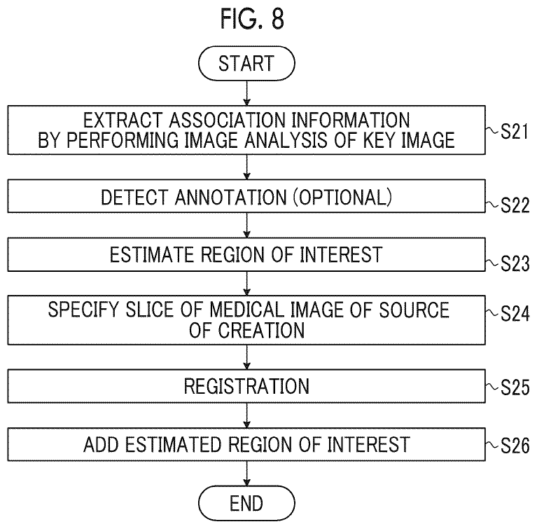

FIG. 8 is a flowchart illustrating a medical image analysis method according to a third embodiment.

Step S21 is the same as step S11 of the second embodiment. Step S22 is the same as step S12 of the second embodiment.

In step S23, the region-of-interest specifying unit 42 specifies the region of interest in the key image acquired in step S21. The region-of-interest specifying unit 42 specifies the region of interest using the region-of-interest estimation model 42A.

Step S24 is the same as step S13 of the second embodiment. Step S25 is the same as step S14 of the second embodiment.

In step S26, the region-of-interest specifying unit 42 adds the region of interest in the key image specified in step S23 to the slice image specified in step S24 and specifies the added region of interest as the region of interest in the slice image. The registration in step S25 enables the region-of-interest specifying unit 42 to add the region of interest to the same position of the slice image as the position of the region of interest in the key image.

As described above, the region of interest in the medical image may be specified by adding the region of interest specified in the key image to the medical image.

### Method of Estimating Corresponding Position: Fourth Embodiment

The region-of-interest specifying unit 42 estimates the position corresponding to the key image in the medical image of the source of creation of the key image based on the association information. A method of estimating the corresponding position will be described.

In a case where the series number is extracted as the association information from the key image by the association information extraction unit 34, the region-of-interest specifying unit 42 specifies a series of the medical image of the source of creation of the key image. In a case where the series number cannot be extracted, the region-of-interest specifying unit 42 specifies the series of the medical image of the source of creation of the key image by searching all series.

In a case where the slice number can be extracted from the key image by the association information extraction unit 34, the region-of-interest specifying unit 42 specifies a slice position of the medical image of the source of creation. In a case where the slice number cannot be extracted from the key image, the region-of-interest specifying unit 42 specifies the slice position of the medical image of the source of creation by searching all slices.

The region-of-interest specifying unit 42 estimates the window level and the window width from the key image. The region-of-interest specifying unit 42 may estimate the window level and the window width from the key image using a window level/window width estimation model (not illustrated) to which a CNN is applied.

The registration unit 44 normalizes the image of the source of creation of the key image using the window level and the window width estimated by the region-of-interest specifying unit 42. Finally, the registration unit 44 estimates the corresponding positions using a general non-rigid registration technique. The non-rigid registration technique includes rotation, translation, and scaling.

### Method of Training Region-Of-Interest Estimation Model: Fifth Embodiment

The region-of-interest specifying unit estimates the region of interest using the region-of-interest estimation model 42A. A method of training the region-of-interest estimation model 42A will be described.

First, the user prepares a medical image in which the position of the region of interest is known, and creates a training medical image from the medical image.

For example, the training medical image is an image obtained by cropping an edge part region of the region of interest in the medical image. The training medical image may be an image obtained by assigning a rectangle to the region of interest in the medical image. In creating the key image, a rectangle larger than a size of the region of interest is generally assigned. Thus, a size of the rectangle is preferably set considering this. The training medical image may be an image obtained by assigning an arrow to the region of interest in the medical image. The training medical image may be a two-dimensional image such as the key image.

A model that estimates the region of interest in the original medical image from the created training medical image, that is, a model that solves an inverse problem, is trained. Accordingly, the region-of-interest estimation model 42A can be created.

That is, the region-of-interest estimation model 42A is obtained by performing machine learning using a training data set for training including a set of the training medical image and the region of interest in the original image of the training medical image. In a case where a cropped image, an image to which a rectangle is assigned, and an image to which an arrow is assigned are provided as input, the region-of-interest estimation model 42A outputs the region of interest in the input image.

The region-of-interest estimation model 42A trained in the above manner can estimate the region of interest from the medical image of the source of creation of the key image and the key image.

According to the medical image analysis method, the key image is analyzed using the image recognition technique, the position corresponding to the medical image of the source of creation is estimated, and the region of interest intended by the doctor is specified using the analysis result and analysis of the medical image of the source of creation. Thus, the medical image in which the region of interest is specified can be used as the training data of the learning model that estimates the region of interest from the medical image.

### Other

The image analysis method according to the present embodiment is also applicable to an image other than the medical image. For example, the method can be applied to a technique for acquiring a diagnosis image of a region of interest created from an original image of a social infrastructure facility such as transportation, electricity, gas, and water supply and specifying a region of interest in an image of a source of creation of the diagnosis image.

The technical scope of the present invention is not limited to the scope according to the embodiments. The configurations and the like in each embodiment can be appropriately combined between each of the embodiments without departing from the gist of the present invention.

### Explanation of References

10: medical image analysis system
12: medical image examination apparatus
14: medical image database
16: user terminal apparatus
16A: input device
16B: display
18: interpretation report database
20: medical image analysis apparatus
20A: processor
20B: memory
20C: communication interface
22: network
32: key image acquisition unit
34: association information extraction unit
36: character recognition unit
38: image recognition unit
38A: image recognition model
40: registration result acquisition unit
42: region-of-interest specifying unit
42A: region-of-interest estimation model
44: registration unit
46: annotation addition unit
48: output unit
AN1: annotation
AN2: annotation
IC: coronal image
ID: medical image
IK1: key image
IK2: key image
IZ: enlarged image
S1 to S2, S11 to S16, S21 to S26: step of medical image analysis method

What is claimed is:

1. A medical image analysis apparatus comprising:
at least one processor; and
at least one memory in which an instruction to be executed by the at least one processor is stored,
wherein the at least one processor is configured to:
acquire a key image that is created from a medical image and that includes a region of interest;
extract association information between the key image and the medical image of a source of creation of the key image by analyzing the key image; and
specify the region of interest in the medical image based on the association information.

2. The medical image analysis apparatus according to claim 1,
wherein the at least one processor is configured to:
estimate the region of interest from the key image; and
add the estimated region of interest to the medical image.

3. The medical image analysis apparatus according to claim 1,
wherein the key image includes an annotation indicating the region of interest, and
the at least one processor is configured to:
add the annotation to the medical image; and
specify the region of interest in the medical image based on the added annotation.

4. The medical image analysis apparatus according to claim 3,
wherein the at least one processor is configured to detect the annotation from the key image.

5. The medical image analysis apparatus according to claim 1,
wherein the medical image includes at least one of a two-dimensional static image, a three-dimensional static image, or a video.

6. The medical image analysis apparatus according to claim 1,
wherein the key image is a result of volume rendering created from the medical image.

7. The medical image analysis apparatus according to claim 1,
wherein the at least one processor is configured to extract the association information by analyzing a character in the key image using character recognition, and
the association information includes at least one of a window width, a window level, a slice number, or a series number of the key image.

8. The medical image analysis apparatus according to claim 1,
wherein the at least one processor is configured to extract the association information by performing image recognition of the key image, and
the association information includes at least one of a window width, a window level, or an annotation of the key image.

9. The medical image analysis apparatus according to claim 1,
wherein the at least one processor is configured to extract the association information from a result of registration between the medical image and the key image.

10. The medical image analysis apparatus according to claim 1,
wherein the at least one processor is configured to estimate a position corresponding to the key image in the medical image based on the association information.

11. The medical image analysis apparatus according to claim 1,
wherein the region of interest is at least one of a mask, a bounding box, or a heat map.

12. The medical image analysis apparatus according to claim 1,
wherein the medical image is a digital imaging and communications in medicine (DICOM) image.

13. A medical image analysis method comprising:
acquiring a key image that is created from a medical image and that includes a region of interest;
extracting association information between the key image and the medical image of a source of creation of the key image by analyzing the key image; and
specifying the region of interest in the medical image based on the association information.

14. A non-transitory, computer-readable tangible recording medium on which a program for causing, when read by a computer, the computer to execute the medical image analysis method according to claim 13 is recorded.
